(19) United States
(12) Patent Application Publication
(10) Pub. No.: US 2022/0346538 A1
(43) Pub. Date: Nov. 3, 2022

(54) Lid Hygiene Apparatus and Method

(71) Applicant: Roosevelt Gloyd, III, Spring, TX (US)

(72) Inventor: Roosevelt Gloyd, III, Spring, TX (US)

(21) Appl. No.: 17/730,143

(22) Filed: Apr. 26, 2022

Related U.S. Application Data
(60) Provisional application No. 63/181,234, filed on Apr. 29, 2021.

(51) Int. Cl. A46B 5/04; A46B 1/00; A46B 5/00; A46D 1/00; A61F 9/007

(52) U.S. Cl. CPC A46B 5/04; A46B 1/00; A46B 5/0037; A46D 1/0207; A46D 1/0276; A61F 9/00718; A46B 2200/1006

(57) ABSTRACT

An apparatus for cleaning an ocular adnexa having a finger sole. The finger sole has a sole surface and a sole bottom. An array of bristles is coupled to the sole bottom. A finger strap is coupled to the sole surface.

### CROSS-REFERENCE TO RELATED APPLICATIONS

This application claims the benefit of U.S. Provisional Application No. 63/181,234 filed Apr. 28, 2021.

### BACKGROUND

Dry eye disease is a common condition that occurs when an individual's tears are not able to provide adequate lubrication for their eyes. Tears can be inadequate and unstable for many reasons. For example, dry eyes may occur if the individual does not produce enough tears or if they produce poor-quality tears. This tear instability leads to inflammation and damage of the eye's surface. Also, many individuals may suffer from Blepharitis which is an inflammatory condition of the eye lids and Meibomitis which is an inflammatory condition of the glands in the eyelid. When the glands become inflamed this could cause poor secretion or no secretion of oil that coats the surface of the eye. This lack of oil will cause evaporation of the water component of individual's tears, thus leading to dry eyes.

Maintaining proper ocular surface hygiene is a challenge.

### BRIEF DESCRIPTION OF THE DRAWINGS

FIG. 1 is a profile view of an ocular adnexa cleaning apparatus.

FIG. 2 is a frontal view of an ocular adnexa cleaning apparatus.

FIG. 3 is a rear view of an ocular adnexa cleaning apparatus.

FIG. 4 is a perspective view of an ocular adnexa cleaning apparatus

FIG. 5 is profile view of two ocular adnexa cleaning bristles.

FIG. 6 is a perspective view of an ocular adnexa cleaning apparatus having an elongated handle.

FIG. 7 is a cross-sectional profile view of an enclosed ocular adnexa cleaning apparatus.

FIG. 8 is a profile view of an enclosed ocular adnexa cleaning apparatus.

FIG. 9 is a perspective view of an enclosed ocular adnexa cleaning apparatus.

FIG. 10 is perspective view of an individual using an ocular adnexa cleaning apparatus.

FIG. 11 is a flow chart illustrating the method of using an ocular adnexa cleaning apparatus.

### DETAILED DESCRIPTION

The following detailed description illustrates embodiments of the present disclosure. These embodiments are described in sufficient detail to enable a person of ordinary skill in the art to practice these embodiments without undue experimentation. It should be understood, however, that the embodiments and examples described herein are given by way of illustration only, and not by way of limitation. Various substitutions, modifications, additions, and rearrangements may be made that remain potential applications of the disclosed techniques. Therefore, the description that follows is not to be taken as limiting on the scope of the appended claims. In particular, an element associated with a particular embodiment should not be limited to association with that particular embodiment but should be assumed to be capable of association with any embodiment discussed herein.

Dry eye disease is a common condition that occurs when an individual's tears are not able to provide adequate lubrication for their eyes. Tears can be inadequate and unstable for many reasons. For example, dry eyes may occur if the individual does not produce enough tears or if they produce poor-quality tears. This tear instability leads to inflammation and damage of the eye's surface. In addition, many individuals may suffer from Blepharitis which is an inflammatory condition of the eye lids and glands. In addition, when the glands become infected this could cause the glands from secreting oil that coats the surface of the eye. This lack of oil will cause the water component of an individual's tears from evaporating, and thus drying out. This is known Meibomian gland dysfunction. The oil that does make it out of the glands can be granular (i.e., crusty) or otherwise unhealthy and can cause irritation.

Meibomian gland dysfunction is very common. In the early stages, individuals are often asymptomatic. However, if left untreated this dysfunction can cause or exacerbate dry eye symptoms and eyelid inflammation. Chronically clogged glands eventually become unable to secrete oil which results in permanent changes in the tear film and dry eyes.

The intent of the embodiments described herein is for ocular surface hygiene, that when used correctly, will substantially reduce the prevalence of dry eye disease and ocular surface disease, which are commonly caused by Meibomian gland dysfunction or Blepharitis. Ocular surface is defined herein as an individual's (i.e., a human) eye lids, eye lashes, and all associated glands that when not healthy and clean contribute to ocular surface disease and dry eye symptoms. Further, the apparatus has features that provide ocular debridement by effectively removing harmful foreign matter that may be present on the ocular surface.

Further, the apparatus described herein may be made of silicone or other similar material, such as plastic polymers or synthetic rubber for example. The cleaning apparatus may be flexible, waterproof, durable, cleanable, and gentle on the ocular surface.

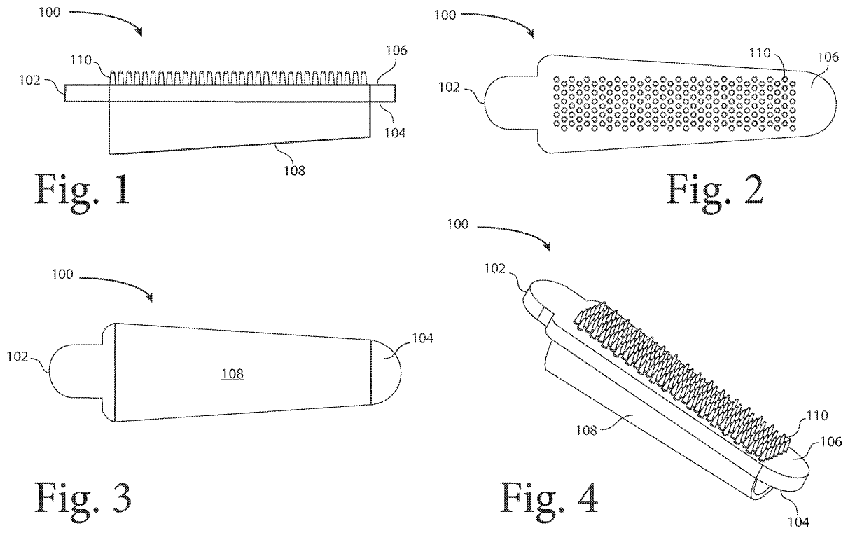

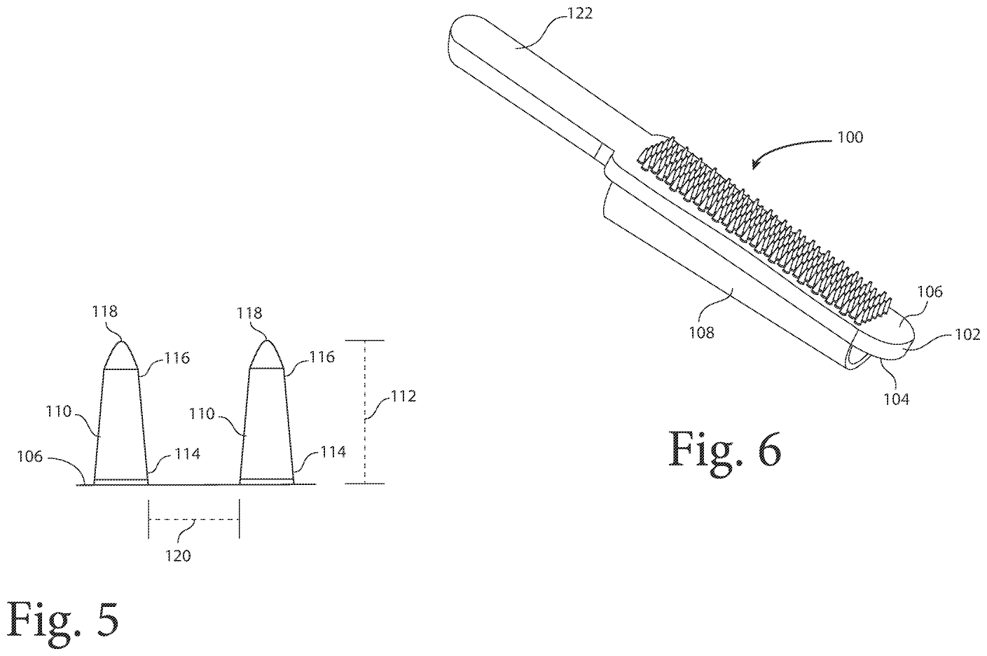

FIGS. 1-4 illustrate different views of an ocular adnexa cleaning apparatus. Specifically, FIG. 1 is a profile view of an ocular adnexa cleaning apparatus. FIG. 2 is a frontal view of an ocular adnexa cleaning apparatus. FIG. 3 is a rear view of an ocular adnexa cleaning apparatus; and FIG. 4 is a perspective view of an ocular adnexa cleaning apparatus. As illustrated in FIGS. 1-4, the ocular adnexa cleaning apparatus 100 may comprise a finger sole 102. The finger sole 102 is a flat elongated surface that is used, in part, as the portion of the ocular adnexa cleaning apparatus 100 that is coupled against an individual's finger. In one or more embodiments, the finger sole 102 has a sole surface 104 and a sole bottom 106 positioned opposite the sole surface 104. The sole surface 104 may include a finger strap 108. The finger strap 108 may be integrally coupled to the sole surface 104 such that it forms a canal for the insertion of an individual's finger. In one or more embodiments, the finger strap 108 is comprised of flexible material so that varying finger sizes may fit securely between the sole surface 104 and the finger strap 108.

In or more embodiments, the sole bottom 106 includes an array of bristles 110 coupled to the sole bottom 106. Note, only one bristle 110 is labeled in the figures for clarity. The small bristles 110 serves as the brush-like surface for the hygiene and debridement of the eyelids, eyelashes, and associated structures. In addition, the array of bristles 110 may vary in size and number and may be positioned in staggered rows and columns.

FIG. 5 is profile view of two ocular adnexa cleaning bristles. As illustrated in FIG. 5, the bristle 110 may have a bristle length 112 of between 0.118 inches and 0.078 inches (i.e., between 3 millimeters and 2 millimeters). The bristle length 112 is to ensure that the bristles 110, while under pressure, does not go beyond an average adult lid margin toward the ocular surface. This limitation in bristle 110 length protects the more aggressive individual from possibly rubbing and traumatizing the ocular surface.

As further illustrated in FIG. 5, the bristle 110 may have a bristle base diameter 114 and a bristle end diameter 116. In one or more embodiments, the bristle base diameter 114 is larger than the bristle end diameter 116 such that the bristle 110 forms a tapered shape. In one or more embodiments, the bristle base diameter 114 has a measurement of between 0.050 inches and 0.025 inches (i.e., between 1.27 millimeters and 0.635 millimeters). In one or more embodiments, the bristle end diameter 116 has a measurement of between 0.040 inches and 0.020 inches (i.e., between 1.016 millimeters and 0.508 millimeters).

The bristle 110 may include a bristle top 118 that forms a conical shape. The tapered and conical shape of the bristle 110 and bristle top 118 is designed to effectively remove, lift, or dislodge stubborn lid debris from an individual's ocular surface.

As further illustrated in FIG. 5, the bristle 110 may have a bristle separation 120 (i.e., the distance between each bristle 110 whether in rows or in columns) of between 0.025 inches and 0.015 inches (i.e., between 0.635 millimeters and 0.381 millimeters). The spacing design of the bristles 110 allows for multiple passes of each of the numerous bristles 110 against the ocular surface. The staggered design from row to row and column to column ensures effectiveness and efficiency as the bristles 110 are moved back and forth across the ocular surface.

FIG. 6 is a perspective view of an ocular adnexa cleaning apparatus having an elongated handle. In one or more embodiments, the ocular adnexa cleaning apparatus 100 may include an elongated handle 122 integrally coupled to the finger sole 102. The elongated handle 122 provides additional leverage for an individual to insert their finger into and between the sole surface 104 and finger strap 108 (e.g., the user will use two fingers from the opposite hand to grip and pull the elongated handle 122 and thus sliding the ocular adnexa cleaning apparatus 100 over the finger of the other hand's finger).

In one or more embodiments, the finger sole 102, the bristles 110, the finger strap 108, and the elongated handle 122 are flexible. In one or more embodiments, the finger sole 102, the bristles 110, the finger strap 108, and the elongated handle 122 are manufactured from a polymer or other similar material. In addition, the finger sole 102, the bristles 110, the finger strap 108, and the elongated handle 122 may have a rubber material hardness of between 30A Shore and 50A Shore.

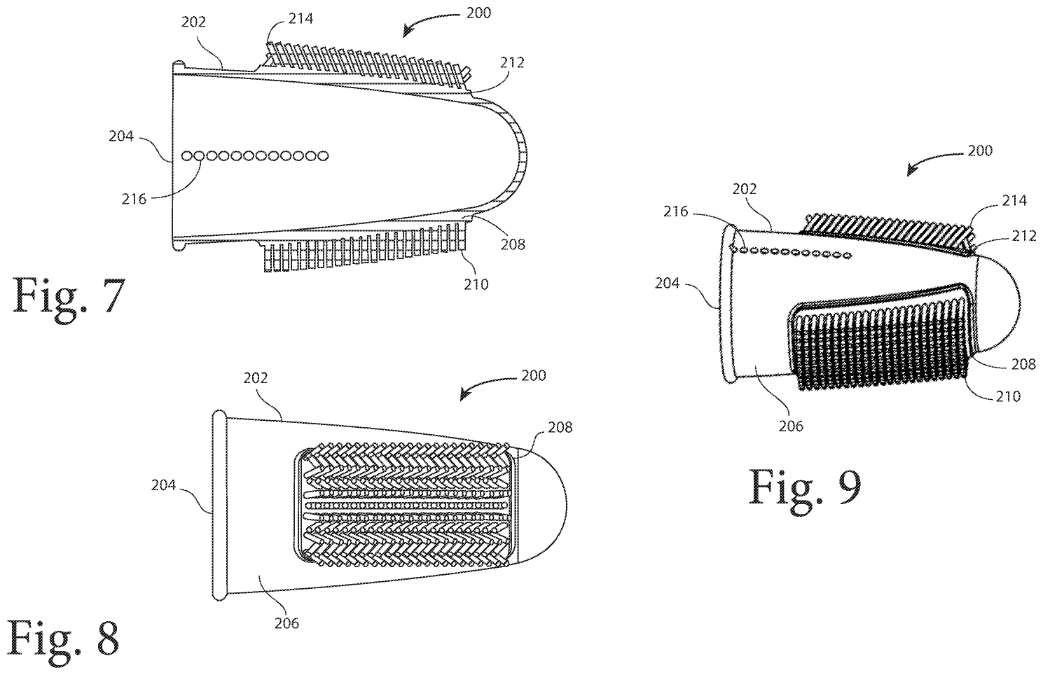

The ocular adnexa cleaning apparatus may have different design variations. For example, the apparatus may form a dome structure with multiple clusters of bristles. FIG. 7 is a cross-sectional profile view of an enclosed ocular adnexa cleaning apparatus. FIG. 8 is a profile view of an enclosed ocular adnexa cleaning apparatus; and FIG. 9 is a perspective view of an enclosed ocular adnexa cleaning apparatus.

As illustrated in FIGS. 7-9, the enclosed ocular adnexa cleaning apparatus 200 may include a finger glove 202. The finger glove 202 may have a domed structure wherein one end of the finger glove 202 is comprised of a rounded enclosure and the opposite end is widened so that it has a larger cavity 204 end. Specifically, the finger glove 202 may have a dome diameter and a base diameter wherein the base diameter is greater than the dome diameter. This design shape of the finger glove 202 allows the individual's finger to be inserted into the open end of the finger glove 202 such that it will fit firmly while being used.

In one or more embodiments, the finger glove 202 includes an outer surface 206. The outer surface 206 may include a first pad 208 integrally coupled to the outer surface 206. In one or more embodiments, the first pad 208 may include a first array of bristles 210 integrally coupled to the first pad 208. Note, only one bristle 210 is labeled in the figures for clarity.

As illustrated in FIGS. 7 and 9, the outer surface 206 may include a second pad 212 integrally coupled to the outer surface 206. In one or more embodiments, the second pad 212 may include a second array of bristles 214 integrally coupled to the second pad 212. Note, only one bristle 214 is labeled in the figures for clarity.

In one or more embodiments, the first array of bristles 210 is integrally coupled to the first pad 208 at an angle of substantially thirty degrees (i.e., within one, three, or five degrees). In one or more embodiments, the second array of bristles 214 is integrally coupled to the second pad 212 at an angle of substantially thirty degrees (i.e., within one, three, or five degrees).

As illustrated in FIGS. 7 and 9, the outer surface 206 may include an array of bore holes 216. In addition, the finger glove 202, the first pad 208, the second pad 212, the first array of bristles 210, and second array of bristles 214 may be flexible. In one or more embodiments, the finger glove 202, the first pad 208, the second pad 212, the first array of bristles 210, and second array of bristles 214 may be manufactured from silicone or other similar material, such as plastic polymers or synthetic rubber for example.

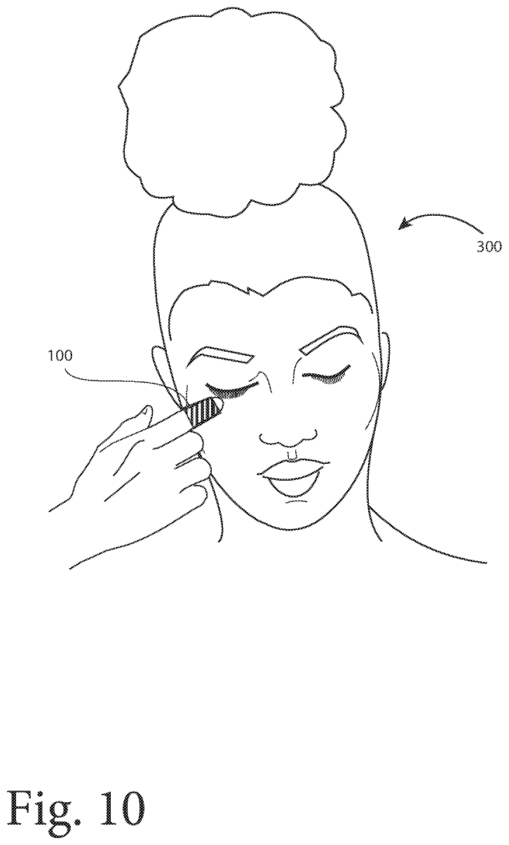

Both embodiments (i.e., ocular adnexa cleaning apparatus 100 and enclosed ocular adnexa cleaning apparatus 200) are used similarly. FIG. 10 is perspective view of an individual using an ocular adnexa cleaning apparatus. FIG. 1l is a flow chart illustrating the method of using an ocular adnexa cleaning apparatus.

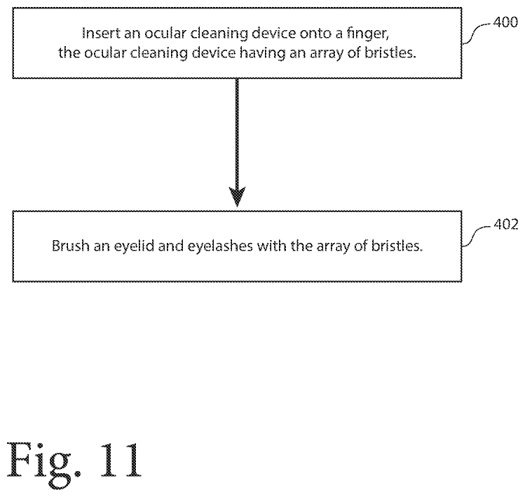

In operation, as illustrated in FIG. 11, an individual 300 (i.e., a human person) inserts an ocular cleaning device (such as ocular cleaning apparatus 100 and enclosed ocular cleaning apparatus 200) onto a finger, the ocular cleaning device (such as ocular cleaning apparatus 100 and enclosed ocular cleaning apparatus 200) having an array of bristles (such as array of bristles 110, first array of bristles 210, and second array of bristles 214) (block 400). Brush the eyelid and eyelashes (such as ocular surface) with the array of bristles (such as array of bristles 110, first array of bristles 210, and second array of bristles 214) (block 402).

In one aspect, the ocular adnexa cleaning device includes a finger sole having a sole surface and a sole bottom. The ocular adnexa cleaning device includes an array of bristles coupled to the sole bottom and a finger strap coupled to the sole surface.

Implementations may include one or more of the following. An elongated handle may be coupled to the finger sole. The finger sole, the bristle, the finger strap, and the elongated handle may be flexible. The finger sole, the bristles, the finger strap, and the elongated handle may be manufactured from a polymer. The bristles may include a bristle base having a bristle base diameter (BB_{D}), a bristle end having a bristle end diameter (BE_{D}), wherein the bristle base diameter (BB_{D}) may be larger than the bristle end (BE_{D}). The bristle end may form a conical shape. The bristle end may have a length of 2.54 millimeters. The bristles may have a rubber material hardness of between 30A Shore and 50A Shore. The bristles may be positioned in staggered rows and columns. The bristles may have a separation distance of 0.51 millimeters.

In one method, the ocular adnexa cleaning device includes inserting an ocular cleaning device onto a finger. The ocular cleaning device has a finger sole having a sole surface and a sole bottom. The ocular adnexa cleaning device includes an array of bristles coupled to the sole bottom and a finger strap coupled to the sole surface. An eyelid and eyelashes are brushed with the array of bristles.

Implementations may include one or more of the following. An elongated handle may be coupled to the finger sole. The finger sole, the bristle, the finger strap, and the elongated handle may be flexible. The finger sole, the bristles, the finger strap, and the elongated handle may be manufactured from a polymer. The bristles may include a bristle base having a bristle base diameter (BB_{D}), a bristle end having a bristle end diameter (BE_{D}), wherein the bristle base diameter (BB_{D}) may be larger than the bristle end (BE_{D}). The bristle end may form a conical shape. The bristle end may have a length of 2.54 millimeters. The bristles may have a rubber material hardness of between 30A Shore and 50A Shore. The bristles may be positioned in staggered rows and columns. The bristles may have a separation distance of 0.51 millimeters.

The operations of the flow diagrams are described with references to the systems/apparatus shown in the block diagrams. However, it should be understood that the operations of the flow diagrams could be performed by embodiments of systems and apparatus other than those discussed with reference to the block diagrams, and embodiments discussed with reference to the systems/apparatus could perform operations different than those discussed with reference to the flow diagrams.

The word “coupled” herein means a direct connection or an indirect connection.

The text above describes one or more specific embodiments of a broader invention. The invention also is carried out in a variety of alternate embodiments and thus is not limited to those described here. The foregoing description of an embodiment of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

What is claimed is:

1. An apparatus for cleaning an ocular adnexa comprising:
a finger sole having:
a sole surface; and
a sole bottom;
an array of bristles coupled to the sole bottom; and
a finger strap coupled to the sole surface.

2. The apparatus of claim 1 further comprising an elongated handle coupled to the finger sole.

3. The apparatus of claim 2 wherein the finger sole, the bristles, the finger strap, and the elongated handle are flexible.

4. The apparatus of claim 2 wherein the finger sole, the bristles, the finger strap, and the elongated handle are manufactured from a polymer.

5. The apparatus of claim 1 wherein the bristles further comprise:
a bristles base having a bristle base diameter (BB_{D}),
a bristle end having a bristle end diameter (BE_{D}); and
wherein the bristle base diameter (BB_{D}) is larger than the bristle end diameter (BE_{D}).

6. The apparatus of claim 5 wherein the bristle further comprises a bristle top that forms a conical shape.

7. The apparatus of claim 1 wherein the bristles have a length of 2.54 millimeters.

8. The apparatus of claim 1 wherein the bristles have a rubber material hardness of between 30A Shore and 50A Shore.

9. The apparatus of claim 1 wherein the array of bristles is positioned in staggered rows and columns.

10. The apparatus of claim 1 wherein the bristles have a separation distance of 0.51 millimeters.

11. A method for cleaning an ocular adnexa comprising:
inserting an ocular cleaning device onto a finger, the ocular cleaning device having:
a finger sole having:
a sole surface; and
a sole bottom;
an array of bristles coupled to the sole bottom; and
a finger strap coupled to the sole surface;
brushing an eyelid and eyelashes with the array of bristles.

12. The method of claim 11 further comprising an elongated handle coupled to the finger sole.

13. The method of claim 12 wherein the finger sole, the bristles, the finger strap, and the elongated handle are flexible.

14. The method of claim 12 wherein the finger sole, the bristles, the finger strap, and the elongated handle are manufactured from a polymer.

15. The method of claim 11 wherein the bristles further comprise:
a bristles base having a bristle base diameter (BB_{D}),
a bristle end having a bristle end diameter (BE_{D}); and
wherein the bristle base diameter (BB_{D}) is larger than the bristle end diameter (BE_{D}).

16. The method of claim 15 wherein the bristle further comprises a bristle top that forms a conical shape.

17. The method of claim 11 wherein the bristles have a length of 2.54 millimeters.

18. The method of claim 11 wherein the bristles have a rubber material hardness of between 30A Shore and 50A Shore.

19. The method of claim 11 wherein the array of bristles is positioned in staggered rows and columns.

20. The method of claim 11 wherein the bristles have a separation distance of 0.51 millimeters.
